(19) United States
(12) Patent Application Publication
(10) Pub. No.: US 2026/0364367 A1
(43) Pub. Date: Nov. 19, 2026

(54) ARGON AND/OR HELIUM COLD PLASMA DEVICE FOR TREATING SKIN AND MUCOUS MEMBRANES, NAILS, HAIR, TEETH AND GUMS

(71) Applicant: FUSION GLOBAL TEKNOLOJI LIMITED SIRKETI, Antalya (TR)

(72) Inventor: Emrah Seyfi TATAR, Antalya (TR)

(73) Assignee: FUSION GLOBAL TEKNOLOJI LIMITED SIRKETI, Antalya (TR)

(21) Appl. No.: 19/487,488

(22) PCT Filed: Jul. 1, 2024
(86) PCT No.: PCT/TR2024/050740

§ 371 Date: Nov. 25, 2025

(30) Foreign Application Priority Data
Jul. 11, 2023 (TR) 2023/008123

(51) Int. Cl. A61N 1/44; A61N 1/05

(52) U.S. Cl. CPC A61N 1/44; A61N 1/0548

(57) ABSTRACT

An argon and/or helium-powered cold plasma device is provided for treating nails, hair, skin and mucous membranes, teeth and gums, including a plastic pen probe, an air connector, a needle electrode, at least one tank, an air hose, a needle hub holder, and a hub connection point, a probe adapter, a general application probe, a nail application probe, a hair application probe, a general dental application probe, a roller application probe and a dental canal application probe.

### CROSS REFERENCE TO THE RELATED APPLICATIONS

This application is the national phase entry of International Application No. PCT/TR2024/050740, filed on Jul. 1, 2024, which is based upon and claims priority to Turkish Patent Application No. 2023/008123, filed on Jul. 11, 2023, the entire contents of which are incorporated herein by reference.

### TECHNICAL FIELD

The present invention relates to a cold plasma device using argon and/or helium for treating nails, hair, skin and mucous membranes, teeth and gums.

### BACKGROUND

Today, the use of cold plasma devices for disinfecting objects or treating skin is well known. Cold plasma combines reactive species such as nitrogen, oxygen, ozone, nitric oxide, ozone, nitric oxide, which inactivate bacteria, viruses and fungi.

Devices in the known state of the art use ambient gases to generate a cold plasma for treating said nails, hair, skin and mucous membranes, teeth and gums, and aim to treat said nails, hair, skin and mucous membranes, teeth and gums with said plasma. However, all these methods do not have the ability to penetrate the nails, hair, skin and mucous membranes, teeth and gums because they do not use any conductive gas in the system and do not have a manipulator to effectively treat the nails, hair, skin and mucous membranes, teeth and gums with the cold plasma produced with these gases. Some of the inventions related to cold plasmas are given below.

Patent application CN105339048A discloses “Cosmetic Use of Cold Plasma”. The invention mentioned in the state of art relates to the cosmetic use of cold atmospheric pressure plasma for the prevention and/or treatment of scalp disorders, in particular aesthetic disorders of the scalp. The invention utilizes cold atmospheric plasma for the prevention and/or treatment of aesthetic disorders of the healthy scalp, in particular dandruff of the healthy scalp. Various gases such as air, helium, oxygen, etc. can be used in the cold atmospheric plasma of the invention.

Patent application US20210401480A1 discloses “Devices and Methods for Treating Skin Tissue Using Cold Plasma”. The invention relates to devices and methods for treating dermatologic diseases and disorders, including skin and nail conditions, using cold plasma. The tube including the invention may contain an inert gas (such as neon or argon) and/or air at sub-atmospheric pressures of at least about 2 kPa or higher.

Patent application US20190105506A1 discloses “Cold Plasma Device for Skin Treatment”. The cold plasma device includes a cold plasma generator configured to generate cold plasma that produces reactive species for treating the skin, and a manipulator configured to manipulate the skin to increase exposure of bacteria on the skin to said reactive species during use of the device.

Patent application US2023056196 discloses “Dental disease treating device and method for treating dental disease”. The device includes a handpiece supplied with gas (e.g. argon) and a tip structure attached to the distal end of the handpiece, which delivers the plasma to a local region and limits the ingress of ambient oxygen in order to reduce ozone formation. The document further experimentally investigates the influence of different tip geometries and gas flow parameters on plasma density and microbial sterilisation performance.

U.S. Pat. No. 4,901,719A discloses “Electrosurgical conductive gas stream equipment”. The system includes an electrosurgical generator, gas delivery apparatus and a hand-held pencil with a nozzle and needle-like electrode, which together form an ionised gas stream that carries the electrical energy to the tissue surface to achieve coagulation (fulguration or desiccation). The document further describes specific structural features of the nozzle, hose and connector that improve gas sealing, increase exposure of the electrode within the gas mixing chamber and allow rotation of the hose without gas leakage.

U.S. Pat. No. 9,554,843 discloses “Adapter and method for converting gas-enhanced electrosurgical coagulation instrument for cutting”. The system includes an electrosurgical generator, gas delivery apparatus and a hand-held pencil with a nozzle and needle-like electrode, which together form an ionised gas stream that carries the electrical energy to the tissue surface to achieve coagulation (fulguration or desiccation).

Patent application FR1340509A discloses a safety device for high-frequency surgical apparatus, which controls the flow of a protective gas around the active electrode and interrupts the high-frequency power supply when this gas is absent. The device includes a gas bottle, a supply line leading to the electrode handle and an electrical sensing element, such as a temperature-dependent resistor arranged in the handle, whose resistance changes with the protective gas flow and is evaluated by an electronic control circuit including a delay stage and switching relays.

Patent application WO2022246070 discloses “Needle adaptor for plasma generating handpiece related application”. The adaptor includes a body with a proximal handpiece coupler, a distal needle connector, an internal energy conductor having fluid passageways, and a fluid line connector, arranged so that plasma-generating energy and an ionisable gas or liquid-gas mixture are conveyed to the needle tip.

Cold plasma devices are described in the patent applications mentioned above. However, when the manipulators discussed in the relevant patent applications are examined structurally, they are insufficient for applications such as skin and mucosa, teeth and gums, dermaroller, hair and nails. Also, inventions mentioned in these documents can not protect the needle from mechanical damage when changing an application probe.

As a result, there is a need for a new device with structural features that can overcome the disadvantages mentioned above and allow more effective shooting or application during applications such as skin, skin and mucous membranes, teeth and gums, dermaroller, hair and nails in manipulators integrated into cold plasma devices using argon and/or helium gas,

### SUMMARY

The present invention is a cold plasma device powered by argon and/or helium for treating nails, hair, skin and mucous membranes, teeth and gums. The present invention is a new device that it has structural features that allow for more effective shooting or application during applications such as skin, skin and mucous membranes, teeth and gums, dermaroller, hair and nails in manipulators integrated in cold plasma devices using argon and/or helium gas.

The invention includes an argon cold plasma generator adapted to generate cold plasma generating reactive species, ozone, nitric oxide, nitrogen gases for treating hair and scalp, and a manipulator adapted to manipulate hair and scalp while exposing said hair and scalp to said reactive species, ozone, nitrogen, nitric oxide gases during use of the device. Said manipulator includes a cold plasma hand probe and a needle electrode at the end of the cold plasma probe designed to contact the scalp during the use of the argon cold plasma device, and a needle electrode at the end of the cold plasma probe designed to trap the argon gas therein while maintaining a balance of ambient gas and argon to treat the hair and scalp without causing damage. Argon-generated cold atmospheric plasma has no cytotoxicity in human skin cells and promotes hair growth by increasing capillary formation, cellular proliferation and protein expression associated with hair/angiogenesis, blood oxygenation and regional blood supply. In addition, argon cold plasma application promotes hair growth through adipogenesis and activation of CD34+CD44+ stem cells and improves the follicular macroenvironment through increased perifollicular vascularity in the hair follicle. Considering the importance of the hair follicle (SF) cycle rate (growth-regression rest) in the diagnosis of alopecia, argon cold atmospheric plasma treatment improves hair growth by increasing the anagen: catagen: telogen ratio by increasing the stem cell activity of (SF). Activation of perifollicular adipose tissue and angiogenesis occurs as a result of upregulation of Wnt/β-catenin signaling and SF regeneration. Finally, these results suggest that argon cold plasma can enhance cellular activities of human skin cells and endothelial cells in vitro and stem cells. Thus, it can enhance angiogenesis, adipogenesis and hair growth in the dermis of the skin. In addition, the inventive device may be a highly effective alternative in regenerative medicine to increase hair growth thanks to the effective application of the argon cold plasma manipulator.

Another manipulator of the invention is an argon cold plasma device for treating foot and nail fungus, which is connected to an argon cold plasma generator. It includes an argon cold plasma generator adapted to generate cold plasma generating reactive species, ozone, nitric oxide, nitrogen gases for treating the nail and foot skin, and a manipulator adapted to manipulate the nail and foot skin while exposing said nail and foot skin to said reactive species, ozone, nitrogen, nitric oxide gases during use of the device.

Anthropophilic dermatophytes are more common than zoophilie fungi as etiologic factors of onychomycoses. In the case of the latter, reverse zoonoses are possible, posing a threat to the persistence of dermatophytes in the environment. However, without treatment, both types of tinea unguium can lead to complete nail plate destruction and secondary mixed infections with fungi and bacteria. One of the zoophilic dermatophytes causing onychomycosis is Trichophyton verrucosum, whose prevalence has increased in recent years. Such infections are usually treated with allylamines and/or azoles. However, such conventional treatment of infections caused by T. verrucosum often fails or is discontinued by patients. In vitro studies on the direct application of argon cold atmospheric plasma on Trichophyton verrucosum growth, germination and nail adhesion as a new alternative treatment for this type of dermatomycosis are presented here. The manipulator referred to herein, the cold plasma hand probe and the needle electrode at the end of the cold plasma probe designed to contact the nail and foot skin during the use of the argon cold plasma device, and the needle electrode at the end of the cold plasma probe designed to contact the nail and foot skin during the use of the argon cold plasma device, trapping the argon gas inside, while at the same time maintaining the balance of ambient gas and argon, allowing the nail and foot skin to be treated without damaging it. In this way, it is possible to popularize the application of argon cold plasma with the inventive nail manipulator and make such treatments more accessible.

Another manipulator of the invention is a helium cold plasma operated dermaroller manipulator. It includes a helium cold plasma generator adapted to generate cold plasma generating reactive species, ozone, nitric oxide, nitrogen gases for treating hair and scalp, and a manipulator adapted to manipulate hair and scalp while exposing said hair and scalp to said reactive species, ozone nitrogen nitric oxide gases during use of the device. Said manipulator operates using a cold plasma hand probe and a dermaroller manipulator at the end of the cold plasma probe designed to contact the scalp during use of the helium cold plasma device and helium gas. The helium cold plasma produced passes through the gaps on the dermaroller and reaches the titanium needles of the dermaroller manipulator, so that the helium cold plasma energy can reach deeper into the skin during the procedure, providing more effective application option.

Another manipulator of the invention is a helium cold plasma tooth, root, canal manipulator. It includes a helium cold plasma generator adapted to generate helium cold plasma producing reactive species, ozone, nitric oxide, nitrogen gases for treating the tooth root and canal, and a manipulator adapted to manipulate the tooth root and canal while exposing said tooth root and canal to said reactive species, ozone, nitrogen, nitric oxide, gases during use of the device. Thanks to this manipulator, helium cold plasma, which previously could not be delivered to the tooth root and canal by any method, is effectively delivered to the tooth root and canal thanks to this manipulator, and it is aimed to combat biofilms in the tooth root and canal more effectively.

Another manipulator of the invention is a helium cold plasma teeth, gums and dental implant manipulator. It includes a helium cold plasma generator adapted to generate helium cold plasma producing reactive species, ozone, nitric oxide, nitrogen gases for treating teeth, gums and dental implants, and a manipulator adapted to manipulate teeth, gums and dental implants while exposing said teeth, gums and dental implants to said reactive species, ozone, nitrogen, nitric oxide, gases during use of the device. Thanks to this manipulator, the helium cold plasma generator can be used to treat areas in the mouth that could not be treated with the helium cold plasma generator before, thanks to the ergonomic design and practicality of the manipulator, even the narrowest points can be treated. Thanks to the invention, it has become possible to deliver helium cold plasma to every area in the mouth. In this way, procedures such as wound healing, stopping bleeding, teeth whitening, infection removal, increasing the tissue compatibility of implants, increasing the bond strength of adhesives can be performed more effectively.

The invention provides a probe adaptor which enables different application probes, such as a general-purpose probe and a roller probe, to be repeatedly exchanged on the same needle while the adaptor mechanically shields the needle from damage during each probe change. By structurally separating the adaptor from the application probe and configuring it to remain fixed on the needle, the invention prevents bending, blunting or misalignment of the needle that would otherwise occur when the probe is handled, attached or removed, thereby reducing the need for premature needle replacement, maintaining sterility and treatment accuracy, and allowing flexible use of multiple probe types in cold plasma therapy with improved safety and reliability.

The structural and characteristic features and all advantages of the product subject to the invention will be more clearly understood thanks to the figures given below and the detailed description written by referring to these figures, and therefore, the evaluation should be made by considering these figures and detailed description.

### BRIEF DESCRIPTION OF THE DRAWINGS

The invention will be described with reference to the accompanying figures, so that the features of the invention will be more clearly understood and appreciated, but the purpose of this is not to limit the invention to these certain regulations. On the contrary, it is intended to cover all alternatives, changes and equivalences that can be included in the area of the invention defined by the accompanying claims. The details shown should be understood that they are shown only for the purpose of describing the preferred embodiments of the present invention and are presented in order to provide the most convenient and easily understandable description of both the shaping of methods and the rules and conceptual features of the invention. In these drawings;

FIG. 1 The general appearance of the inventive product.

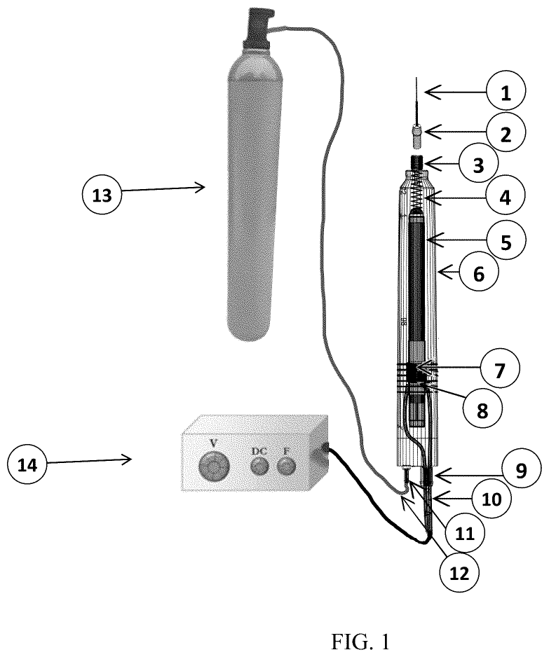

FIG. 2 Detail view of the probes of the inventive product.

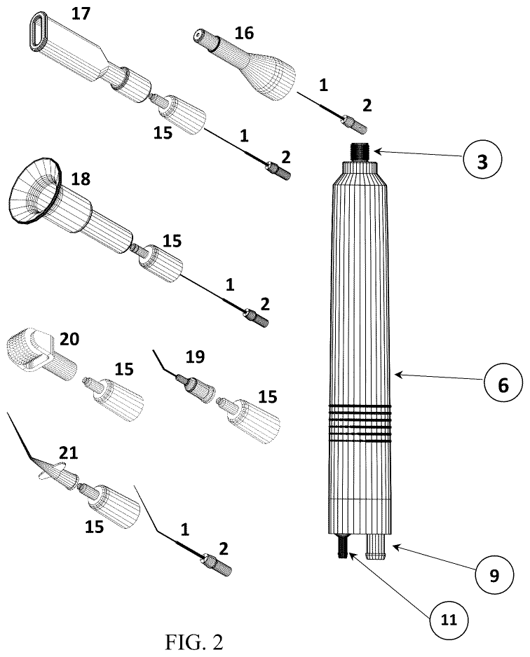

The figures to help understand the present invention are numbered as indicated in the attached image and are given below along with their names.

### DESCRIPTION OF REFERENCES

1. Needle electrode
2. Needle hub holder
3. Hub connection point
4. Core connection spring
5. Core coil
6. Plastic pen probe
7. Core coil winding
8. High voltage and core connection point
9. High voltage probe input
10. High voltage cable
11. Air connector
12. Air hose
13. Tank
14. High voltage generator
15. Probe adapter
16. General application probe
17. Nail application probe
18. Hair application probe
19. General dental practice probe
20. Roller application probe
21. Root canal application probe

### DETAILED DESCRIPTION OF THE EMBODIMENTS

The invention includes at least one tank (13) in which argon and/or helium gas is stored, an air hose (12) for conveying argon and/or helium gas contained in the tank (13) by the air connector (11) to the plastic pen probe (6), a needle hub holder (2) associated with the needle electrode (1), hub connection point (3) positioned on the plastic pen probe (6) for fixing the needle hub holder to plastic pen probe (6), the needle electrode (1) and the probe adapter (15) with a perforated structure that can pass through the needle hub holder (2), the roller application probe (20), which is formed in a form that can be attached and removed from the probe adapter (15) and has rotatable rollers at the end and these rollers are fixed at two points and the plasma energy penetrates the roller tips and then the skin through the airways.

In addition to the above embodiment, the invention further includes a general application probe (16) which is mounted on the needle hub holder (2) in order to deliver cold plasma energy to the target tissue at the required temperature without spreading from a single point, and the end part of which has a gradually narrowing structure towards the part to be applied and the end part to contact the tissue has a flat cross-section so that it is not pointed.

In addition to the above embodiment, the invention further includes a nail application probe (17) having an elliptical shape to create a vacuum effect in different devices and to transmit the electric ion current via at least one electrically conductive wire in the cold plasma device and to increase the effect rate by reducing the gas content in the air. This probe has an elliptical tip for optimum plasma distribution over a large area and optimum delivery to the nail surface.

In addition to the above embodiment, the invention further includes a hair application probe (18) having an expanding tip in the form of a cylindrical form, wherein the tip portion has a structure that gradually or non-gradually expands towards the part to be applied in order to increase the rate of effect on the surface to be applied.

In addition to the above embodiment, the invention further includes a general dental application probe (19) having a curved needle at the end thereof, which is formed in a removable form for attachment to the probe adapter (15) for convenient use of the plasma in the teeth and mouth.

In addition to the above embodiment, the invention further includes a root canal application probe (21) including at least two fins, the tip of which has a tapered structure for the use of plasma in root canal treatment in the canal area and in hard-to-reach places in the mouth, formed in a removable form that can be attached to the probe adapter (15).

In all the probes described here, the needle distance provides a plasma conduction difference and provides improvement in the region by working at optimal temperatures according to the shape of the ion flow of the cold plasma and the region where it can work.

What is claimed is:

1-7. (canceled)

8. A cold plasma device powered with argon and/or helium for treating nails, hair, skin and mucous membranes, teeth and gums, comprising a plastic pen probe, an air connector, a needle electrode, at least one tank for storing the argon and/or the helium, an air hose for delivering the argon and/or the helium contained in the at least one tank to the plastic pen probe via the air connector, a needle hub holder associated to the needle electrode, a hub connection point positioned on the plastic pen probe for fixing the needle hub holder to the plastic pen probe,
a probe adapter with a perforated structure, wherein the perforated structure passes through the needle electrode and the needle hub holder, and
a roller application probe, wherein the roller application probe is attachable to and removable from the probe adapter and the roller application probe has rotatable rollers at an end, the rotatable rollers are fixed at two points, and plasma energy penetrates roller tips and then the skin through airways.

9. The cold plasma device according to claim 8, further comprising a nail application probe, wherein the nail application probe is attachable to the probe adapter and has an elliptical shape to create a vacuum effect and to transmit an electric ion current via at least one electrically conductive wire in the cold plasma device and to increase an effect rate by reducing a gas ratio in air.

10. The cold plasma device according to claim 9, wherein the nail application probe has an elliptical tip.

11. The cold plasma device according to claim 8, further comprising a hair application probe, wherein the hair application probe is attachable to the probe adapter and has an expanding tip in a form of a cylindrical form in which the expanding tip has a structure that gradually or non-gradually expands towards a part to be applied in order to increase an effect rate on a surface to be applied.

12. The cold plasma device according to claim 8, further comprising a general dental application probe with a curved needle at a tip, wherein the general dental application probe is attachable to and removable from the probe adapter to ensure comfortable use of plasma in the teeth and a mouth.

13. The cold plasma device according to claim 8, further comprising a root canal application probe with a tapered tip and at least two fins for use of plasma in root canal treatment in a canal area and hard-to-reach places in a mouth, wherein the root canal application probe is attachable to and removable from the probe adapter.
